(19) United States
(12) Patent Application Publication
(10) Pub. No.: US 2022/0284989 A1
(43) Pub. Date: Sep. 8, 2022

(54) IMPLEMENTATION METHOD OF MOLECULAR OMICS DATA STRUCTURE BASED ON DATA INDEPENDENT ACQUISITION MASS SPECTRA

(71) Applicant: Westlake University, Hangzhou (CN)

(72) Inventors: Tiannan Guo, HANGZHOU (CN); Zhongzhi Luan, HANGZHOU (CN); Ziqing Li, HANGZHOU (CN); Fangfei Zhang, HANGZHOU (CN); Shaoyang Yu, HANGZHOU (CN); Zelin Zang, HANGZHOU (CN)

(21) Appl. No.: 17/597,648

(22) PCT Filed: Nov. 10, 2020
(86) PCT No.: PCT/CN2020/127823

§ 371 Date: Jan. 15, 2022

(30) Foreign Application Priority Data
Mar. 4, 2020 (CN) 202010144110.0

(51) Int. Cl. G16C 20/20; G16C 20/70; G16C 20/80; G01N 27/62

(52) U.S. Cl. CPC G16C 20/20; G16C 20/70; G16C 20/80; G01N 27/62

(57) ABSTRACT

The present invention relates to the technical field of biomolecular omics mass spectrometry data, in particular to an implementation method of a molecular omics data structure based on data independent acquisition mass spectra. The mass spectrometry data structure is DIAT (Data-Independent Acquisition Tensor) data generated from original mass spectrometry data and has attributes of three dimensions, the first dimension is a cycle index, the second dimension is a fragment ion mass-to-charge ratio, and the third dimension is a precursor ion window index corresponding to a fragment ion. The DIAT data of this solution is high in integrity, convenient to read and high in reading speed, and the size of a DIAT file is only a few tenths of that of an mzXML file. DIA mass spectrometry data can be directly observed through a visualized pooled DIAT file image, and a DIAT can be analyzed by directly using a visual processing algorithm, which avoids the operation of extracting ion chromatographic with a large amount of calculation and can directly establish a computer deep learning model for clinical phenotype classification and prediction according to the file.

### CROSS-REFERENCE TO RELATED APPLICATIONS

This application is a 371 of International Patent Application Number PCT/CN2020/127823, filed on Nov. 10, 2020, which claims the benefit and priority of Chinese Patent Application Number 202010144110.0, filed on Mar. 4 2020 with China National Intellectual Property Administration, the disclosures of which are incorporated herein by reference in their entireties.

### BACKGROUND OF THE PRESENT INVENTION

### Field of Invention

The present invention relates to the technical field of biomolecular omics mass spectrometry data, in particular to an implementation method of a molecular omics data structure based on data independent acquisition mass spectra.

### Description of Related Arts

Mass spectrometry (MS)-based omics has been developed for decades, and it has been developed to be available for molecular analysis on thousands of biomolecules in complex biological samples within a few hours. Biomolecules are separated by liquid chromatography (LC) and identified and quantified by tandem mass spectrometry (MS/MS). The omics technology includes proteomics, metabolomics and lipidomics.

The mass spectrometry-based omics currently has the following acquisition modes:

1. Data dependent acquisition (DDA): the data dependent acquisition depends on the intensity of precursor ions in MS1 of a sample, and sorting the precursor ions for fragmentation in MS2 has certain randomness, so the identification reproducibility is relatively low;

2. Selected reaction monitoring (SRM): target method-selected reaction monitoring can accurately analyze a limited set of predefined molecules, but the throughput is only of hundreds;

3. Data independent acquisition (DIA): DIA is a holographic data independent acquisition quantitative technology, which divides the entire full scan range of a mass spectra into a number of windows, cyclically selects, fragments and detects all ions in each window at a high speed so as to obtain all fragment information of all ions in the sample without omission and difference, does not need to specify targeted molecules, adopts uniform scanning points, can achieve qualitative confirmation and quantitative ion screening by using a spectral library, and can realize data backtracking. For example: Sequential window acquisition of all theoretical mass spectra (SWATH) divides a MS1 into a series of adjacent precursor ion selection windows of 25 m/z or a larger size. In each window, each precursor ion is fragmented with all other precursor ions at the same time. This technology also records corresponding multiple spectra of fragment ions from the same window. Fragment ions falling into the same precursor ion window can be systematically recorded without bias, which overcomes the randomness of precursor ion selection in the DDA mode and also retains high accuracy of the target method. The data independent acquisition mass spectrometry method can repeatedly cover low-abundance molecules, so that a permanent digital atlas can be generated to represent all measurable molecular signals as a digital archive of biomolecular omics.

In practical applications, most mass spectrometer manufacturers have protected mass spectrometry data formats, such as ThermoFisher's raw format, Sciex's wiff format, and Bruker's baf format. Although there are some open-source converted data formats on the market, such as mzXML format, mzML format, and mz5 format, these formats generally have the problem of low storage efficiency. For example: extensible markup language (XML)-based file formats (such as mzXML format and mzML format) are converted into readable languages and cannot directly store binary data, resulting in a significant increase in the file size of the converted XML format; and the reading of an XML file must be sequential reading, and non-sequential reading of data is required for mass spectrometry data analysis, thus resulting in the problem of low input and output (I/O) rates. Although the Mz5 format is an efficient data management and storage format based on High-performance data management and storage5 (HDF5), it still maintains the ontology of mzML file content, which is not all information required for DIA data analysis. In addition, due to the loss of the relationship between precursor ions and fragment ions in DIA, the precursor ions flowing out together will be fragmented in the same window, producing a highly complex fragment mass spectra. Therefore, it is necessary to obtain prior information of targeted molecules in DDA, including a precursor mass-to-charge ratio, a mass-to-charge ratio of fragment ions, their corresponding relative intensities and retention times, etc., and then extraction of ion chromatograph (XIC) will be performed to infer a peak group belonging to the targeted molecules, which consumes a lot of computing resources and time and often leads to data distortion. Although various existing DIA analysis software, such as OpenSWATH software, Skyline software, Spectronaut software, and PeakView software, can realize the function of identifying and quantifying biomolecules, these programs are not easy to operate and consume a lot of time and computing resources, and only some of the MS2 are used for peak group inference, which will produce unpredictable effects (for example: inevitable missing value problem) to affect downstream statistical classification analysis.

Therefore, the existing mass spectrometry data structure is no longer suitable for storing and analyzing large-scale data generated by the novel data independent acquisition mass spectrometry.

### SUMMARY OF THE PRESENT INVENTION

In response to the problems in the prior art, the present invention provides a biomolecular omics mass spectrometry data structure based on data independent acquisition mass spectra and an implementation method thereof.

In order to achieve the above technical objective, the technical solutions of the present invention are:

1. A molecular omics data structure based on data independent acquisition mass spectra, the mass spectrometry data structure is DIAT (Data-Independent Acquisition Tensor) data generated from original mass spectrometry data, where the DIAT data has attributes of three dimensions, the first dimension is a cycle index, the second dimension is a pooled fragment ion mass-to-charge ratio, and the third dimension is a precursor ion window index corresponding to a fragment ion.

2. An implementation method of a molecular omics data structure based on data independent acquisition mass spectra, including the following steps:

Step A: converting an original mass spectrometry data file into a mzXML format file, and performing centroiding for the original mass spectrometry data, the obtained mzXML format file including all necessary information of MS1 and MS2 data;

Step B: extracting required mass spectrometry data from the mzXML format file obtained in step A, the mass spectrometry data including at least the following attributes: scan level, scan index, retention time, precursor ion mass-to-charge ratio, fragment ion mass-to-charge ratio and fragment ion intensity;

Step C: counting the total number of cycles and cycle indexes for the mass spectrometry data extracted in step B according to the scan level and scan index, performing loss scan detection, filling in 0 placeholders in all lost positions, and obtaining windows and cycle indexes of precursor ions corresponding to fragment ions in the data;

Step D: binning the mass spectrometry data obtained in step C according to the attribute of the fragment ion mass-to-charge ratio, and summing intensity values of fragment ions falling in the same fragment ion mass-to-charge ratio bin;

Step E: reordering the mass spectrometry data processed in step D, wherein the reordering refers to obtaining corresponding window indexes according to the precursor ion mass-to-charge ratio data corresponding to MS2 spectra, and rearranging the MS2 having the same window index in order of cycle indexes; and

Step F: constituting tensor data of MS2 fragment ion intensity from the data processed in step E based on three dimensions: a cycle index, a fragment ion mass-to-charge ratio, and a precursor ion window index corresponding to a fragment ion.

As an improvement, the method further includes step G: pooling the data of different dimensions to reduce the size of the tensor data and then generating pooled DIAT data.

Preferably, the method of pooling in step G is: first, in each precursor isolation window, performing distribution statistical estimation on non-zero values of precursor ion mass-to-charge ratios to obtain a main and sub alternating peak mode with predefined grids; then pooling different mass-to-charge ratio areas by the pattern of the main and sub alternating peak mode, where the upper and lower boundaries of the mass-to-charge ratio areas were determined using nonlinear square Gaussian fitting of non-zero intensity distribution peaks; finally, discarding all grids without peaks, and merging multiple rows of the main and sub peak areas into one row to reduce the rows in the mass-to-charge ratio dimension.

As an improvement, the method further includes the following step: after obtaining the pooled DIAT data, processing the DIAT data into a pseudo-color image to achieve visualization.

As an improvement, the method further includes the following step: after obtaining the pooled DIAT data, graying the fragment ion intensity in the DIAT data as an input model for deep learning.

It can be seen from the above description, that the present invention has the following advantages:

The DIAT data of the present invention is transformed according to the original mass spectrometry data structure, which can ensure the retain of effective information of the DIA mass spectrometry data; and the data is read in the form of a three-dimensional tensor, and the reading sequence is not restricted, which greatly improves the convenience and speed of data reading. After the DIAT data is stored as a DIAT format file, the file size is only a few tenths of that of the mzXML file, which greatly reduces the storage space required for the mass spectrometry data file. The present invention can also directly observe the DIA mass spectrometry data through the visualized pooled DIAT file image, and can directly use the visual processing algorithm to analyze the DIAT, which avoids the performance of extraction of ion chromatographic (XIC) with a large amount of calculation, and can directly establish a computer deep learning model for clinical phenotype classification and prediction according to the format file. With the increase in the quality and quantity of DIA data, the potential of the technology of the present invention in clinical diagnosis can be foreseen, and an effective solution can be provided for classificatory diagnosis of diseases.

### BRIEF DESCRIPTION OF THE DRAWINGS

FIG. 1 is a flowchart of an implementation method of the present invention;

FIG. 2 is a schematic illustration of original mass spectrometry data of the present invention;

FIG. 3 is a schematic illustration of DIAT data after format conversion of the original mass spectrometry data of the present invention;

FIG. 4 is a schematic illustration of a cycle index of the DIAT data of the present invention;

FIG. 5 is a schematic illustration of the DIAT data of the present invention;

FIG. 6 is a size comparison diagram of a DIAT file, an mzXML file and an original mass spectrometry data file in the present invention;

FIG. 7 is a schematic illustration of pooled DIAT data in the present invention;

FIG. 8 is a schematic illustration of main and sub peaks of experimental data of the present invention;

FIG. 9 is a Gaussian distribution fitting diagram of the present invention;

FIG. 10 is a schematic illustration of simulated main peaks of the present invention;

FIG. 11 is a schematic illustration of a visualization process of a two-dimensional graph of the present invention;

FIG. 12 is a schematic illustration of graying results of the present invention applied to proteomic data;

FIG. 13 is a schematic illustration of graying results of the present invention applied to metabolomic data;

FIG. 14 is a schematic illustration of graying results of the present invention applied to lipidomic data.

### DETAILED DESCRIPTION OF THE PREFERRED EMBODIMENT

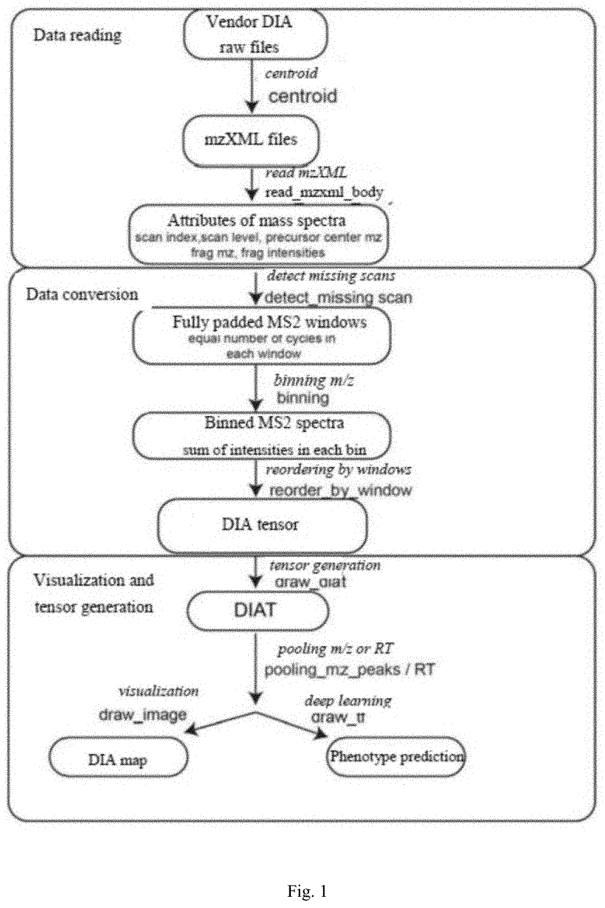

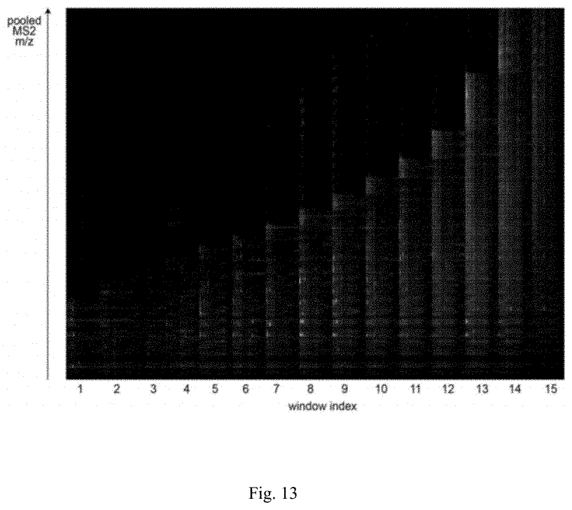

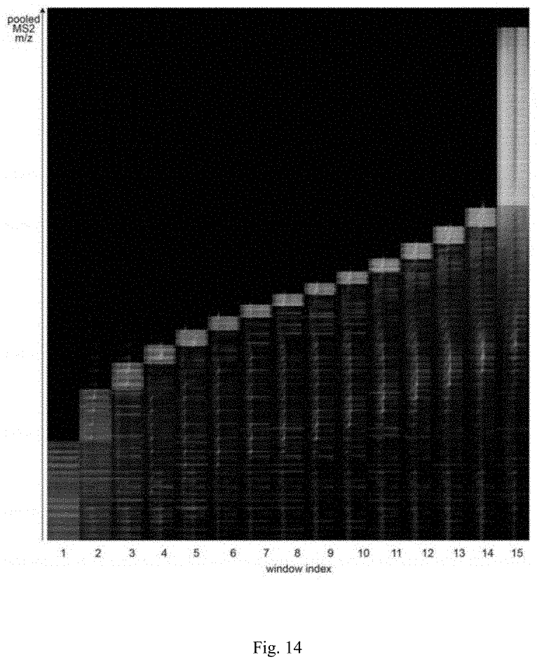

With reference to FIGS. 1 to 14, the embodiments of the present invention are described in detail, but the claims of the present invention are not limited in any way.

As shown in FIG. 1, an implementation method of a biomolecular omics data structure based on data independent acquisition mass spectra includes the following specific steps:

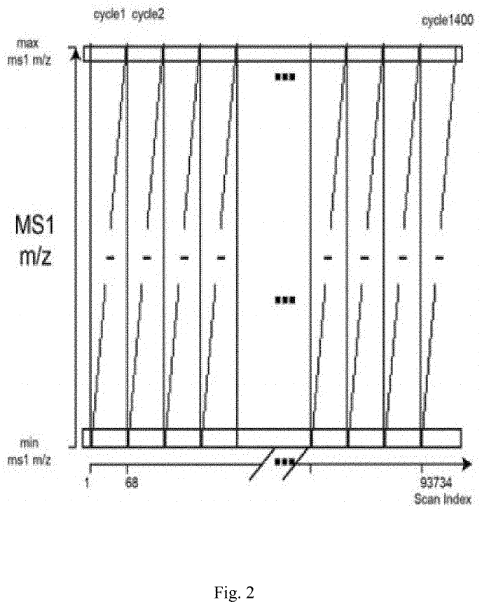

Step A: an original mass spectrometry data file provided by a supplier is converted into a mzXML format file by using the MSconvert tool in the ProteoWizard software package, and performing centroiding for the original mass spectrometry data file by the MSconvert tool, the obtained mzXML format file including all necessary information of MS1 and MS2 data (as shown in FIG. 2, a schematic illustration of the original mass spectrometry data file provided by the supplier);

Step B: a read_mzxml_body function is written, and required mass spectrometry data is extracted from the mzXML format file obtained in step A by using the pyteomic toolkit, the mass spectrometry data at least including the following attributes: scan level (MS level), scan index, retention time, precursor ion mass-to-charge ratio (peptide precursor m/z), fragment ion mass-to-charge ratio (fragment m/z), and fragment ion intensity (fragment intensity);

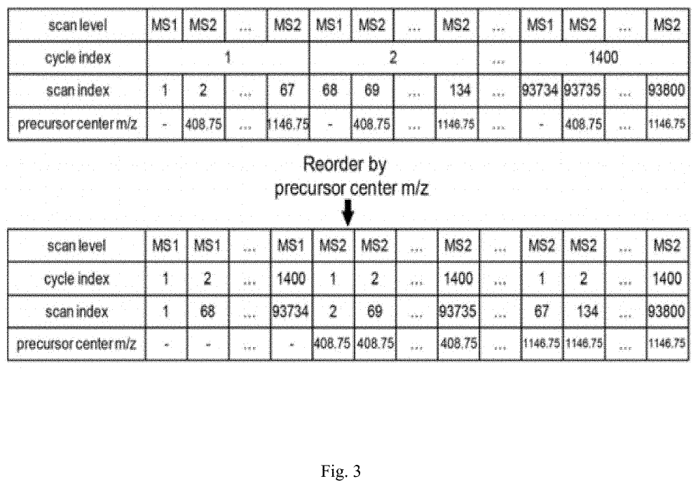

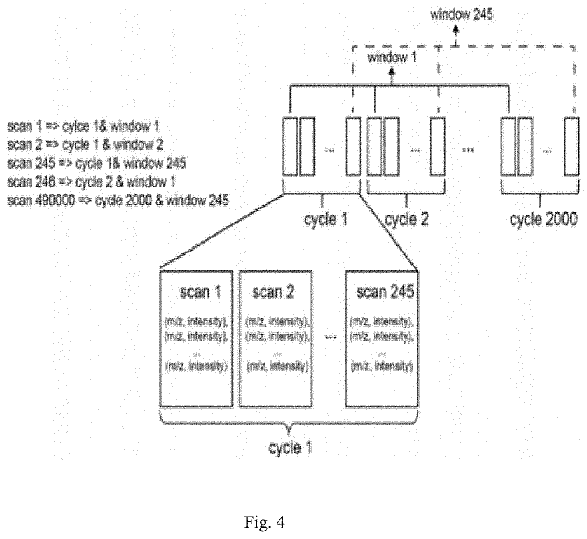

Step C: the total number of cycles and cycle indexes are counted by using a detect_missing_scan function for the mass spectrometry data extracted in step B according to the scan level and scan index (as shown in FIG. 3), loss scan detection is performed at the same time, 0 placeholders are filled in all lost positions, and windows and cycle indexes of precursor ions corresponding to fragment ions in the data are obtained (as shown in FIG. 4);

Step D: the mass spectrometry data obtained in step C is binned by using a binning function according to the attribute of the fragment ion mass-to-charge ratio, and intensity values of fragment ions falling in the same fragment ion mass-to-charge ratio bin are summed, the bin size being set according to the mass accuracy of different mass spectrometry machines, so as not to affect the overall integrity of the data;

Step E: since the original data format of data independent acquisition mass spectra is a repeated cycle formed by a MS1 plus a series of MS2, each MS2 in the same acquisition cycle is relatively independent, and the MS2 corresponding to the same precursor ion mass-to-charge ratio in different cycles are associated each other, so the mass spectrometry data processed in step D is reordered by using a reorder_by_window function, wherein the reordering refers to obtaining corresponding window indexes according to the precursor ion mass-to-charge ratio data corresponding to the MS2, and rearranging the MS2 having the same window index in order of cycle indexes; and

Step F: DIAT (Data-Independent Acquisition Tensor) data of MS2 fragment ion intensity is constituted from the data processed in step E based on three dimensions: a cycle index, a fragment ion mass-to-charge ratio, and a precursor ion window index corresponding to a fragment ion.

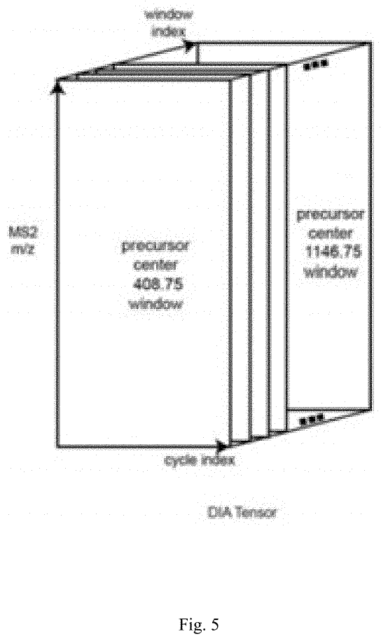

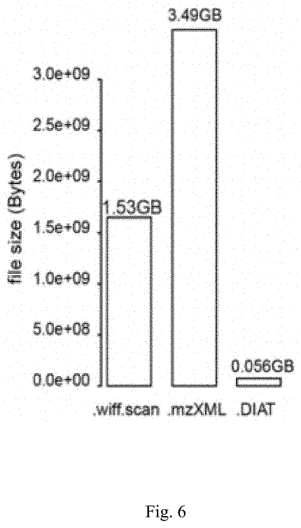

Through the foregoing implementation method, the final result is a biomolecular omics mass spectrometry data structure based on data independent acquisition mass spectra. As shown in FIG. 5, the mass spectrometry data structure is a DIAT data having attributes of three dimensions, the first dimension is a cycle index, the second dimension is a fragment ion mass-to-charge ratio, and the third dimension is a precursor ion window index corresponding to a fragment ion. The DIAT data is transformed according to the original mass spectrometry data structure, which can ensure the retain of effective information of the DIA mass spectrometry data; and the data is read in the form of a three-dimensional tensor, and the reading sequence is not restricted, which greatly improves the convenience and speed of data reading. After the DIAT (Data-Independent Acquisition Tensor) data is stored as a DIAT file (stored in a .diat format), the file size will be reduced to a few tenths of that of the original mzXML file. FIG. 6 shows a size comparison diagram of a DIAT file generated from the example of FIG. 2, an mzXML file and an original mass spectrometry data file. It can be seen from FIG. 6 that the size of the DIAT file is reduced by 30 times compared with the original mass spectrometry data file, and reduced to 1/60 of the size of the mzXML file, which greatly reduces the storage space required for the mass spectrometry data file.

In the above-mentioned implementation method, it should be noted that since the number of cycles in the mzXML file converted from the same batch of original mass spectrometry data may be different, it is necessary to count the total number of cycles of mass spectra in different files, and round the minimum number of cycles in the same batch down by tens as a uniform number of cycles of this batch of data reading, to ensure a uniform number of scans for subsequent data processing.

After the above-mentioned DIAT data is obtained, in order to further improve the performance of the data, the following improvements are made to the above technical solutions:

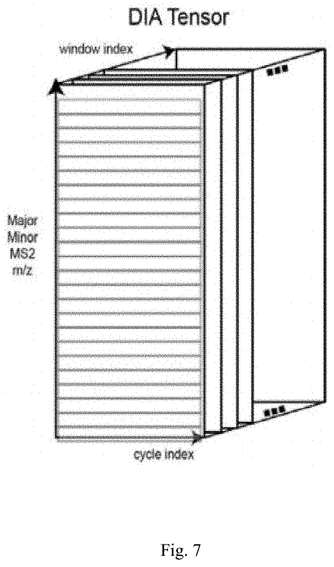

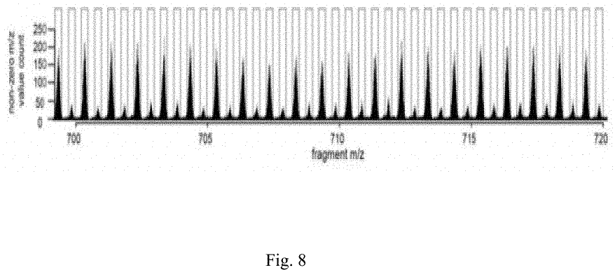

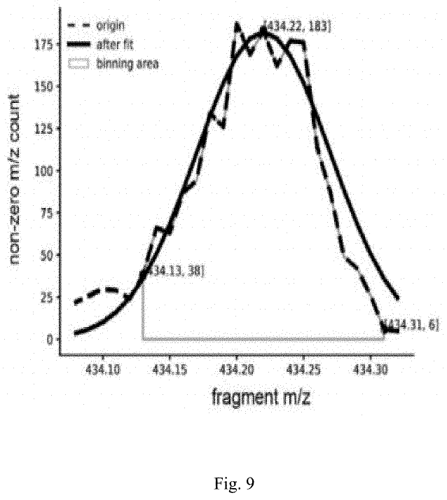

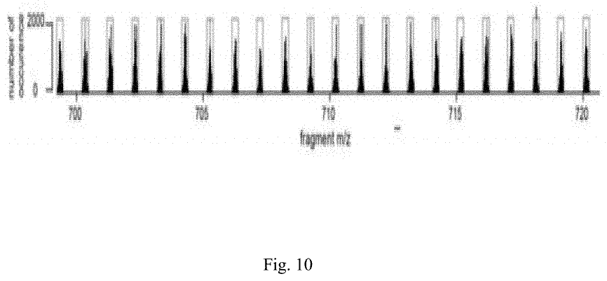

(1) Step G is added: the data of different dimensions is pooled to reduce the size of the tensor data, to generate pooled DIAT data (as shown in FIG. 7, which is a schematic illustration of three-dimensional DIAT data including main and sub peaks). The specific method of pooling may be: first, in each precursor isolation window, distribution statistical estimation is performed on non-zero values of precursor ion mass-to-charge ratios to obtain a main and sub alternating peak mode with predefined grids (as shown in FIG. 8), then different mass-to-charge ratio areas are pooled by the pattern of the main and sub alternating peak mode, upper and lower boundaries of the mass-to-charge ratio areas that need to be merged are dynamically determined by using nonlinear square Gaussian fitting of non-zero intensity distribution peaks (as shown in FIG. 9), finally all grids without peaks are discarded by using a pooling_mz_peaks_by_window function, and multiple rows of the main and sub peak areas are merged into one row to reduce the rows in the mass-to-charge ratio dimension by 50 times. In this step, the main and sub alternating peak mode with predefined grids can be used as the pooling law because the results of simulating the distribution of singly charged fragment ions of all human proteomes (as shown in FIG. 10) have the same main peak distribution mode as the real experimental sample, and the sub peak can be interpreted as the mass-to-charge ratio of doubly charged fragment ions.

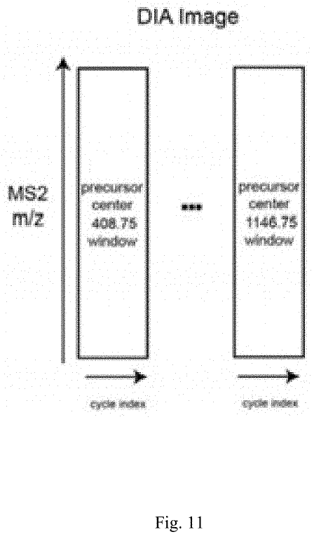

(2) After the pooled DIAT data is obtained, the DIAT data is processed into a pseudo-color image by using a draw_image function to achieve visualization, as shown in FIG. 11, which is a schematic illustration of two-dimensional image visualization. Through the visualization, not only can the DIA mass spectrometry data be directly observed through a visualized DIAT file image, but also can the DIAT be analyzed by directly using a visual processing algorithm, which avoids the performance of extraction of ion chromatographic (XIC) with a large amount of calculation and can directly establish a model for clinical phenotype classification and prediction according to the file.

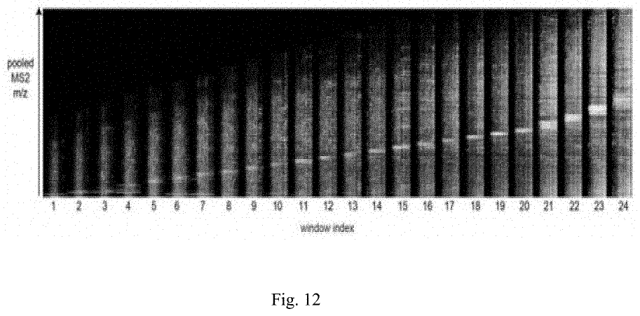

(3) After the pooled DIAT data is obtained, the fragment ion intensity in the DIAT data is grayed by using a draw_diat function as an input model for subsequent deep learning. For example: the method of graying is: equal-frequency discrete division is performed on non-zero values of intensity by using percentiles, and the divided areas are colored. 0 to 100 are divided at equal intervals into 256 values, 256 values corresponding to non-zero values of intensity are calculated by using 256 floating point numbers from 0 to 100 and percentile function, the 256 values correspond to 255 intervals, each interval corresponds to one color, and the interval value ranges from 1 to 255. FIGS. 12-14 shows schematic illustrations of graying results obtained with proteomics, metabolomics and lipidomics as application objects.

In summary, the present invention has the following advantages:

The DIAT data of the present invention is transformed according to the original mass spectrometry data structure, which can ensure the retain of effective information of the DIA mass spectrometry data; and the data is read in the form of a three-dimensional tensor, and the reading sequence is not restricted, which greatly improves the convenience and speed of data reading. After the DIAT data is stored as a DIAT file, the file size is only a few tenths of that of the mzXML file, which greatly reduces the storage space required for the mass spectrometry data file. The present invention can also directly observe the DIA mass spectrometry data through the visualized pooled DIAT file image, and can directly use the visual processing algorithm to analyze the DIAT, which avoids the operation of extracting ion chromatographic (XIC) with a large amount of calculation and can directly establish a computer deep learning model for clinical phenotype classification and prediction according to the format file. With the increase in the quality and quantity of DIA data, the potential of the technology of the present invention in clinical diagnosis can be foreseen, and an effective solution can be provided for classificatory diagnosis of diseases.

It can be understood that the above specific descriptions of the present invention are only used to illustrate the present invention and are not limited to the technical solutions described in the embodiments of the present invention. Those of ordinary skill in the art should understand that the present invention can still be modified or equivalently replaced to achieve the same technical effects; as long as the requirements for use are met, these modifications or equivalent replacements shall fall into the protection scope of the present invention.

1. An implementation method of a molecular omics data structure based on data independent acquisition mass spectra, comprising the following steps:
step A: converting an original mass spectrometry data file into a mzXML format file, and performing centroiding for the original mass spectrometry data, the obtained mzXML format file comprising all necessary information of MS1 and MS2 data;
step B: extracting required mass spectrometry data from the mzXML format file obtained in step A, the mass spectrometry data comprising at least the following attributes: scan level, scan index, retention time, precursor ion mass-to-charge ratio, fragment ion mass-to-charge ratio and fragment ion intensity;
step C: counting the total number of cycles and cycle indexes for the mass spectrometry data extracted in step B according to the scan level and scan index, performing loss scan detection, filling in 0 placeholders in all lost positions, and obtaining windows and cycle indexes of precursor ions corresponding to fragment ions in the data;
step D: binning the mass spectrometry data obtained in step C according to the attribute of the fragment ion mass-to-charge ratio, and summing intensity values of fragment ions falling in the same fragment ion mass-to-charge ratio bin;
step E: reordering the mass spectrometry data processed in step D, wherein the reordering refers to obtaining corresponding window indexes according to the precursor ion mass-to-charge ratio data corresponding to the MS2, and rearranging the MS2 having the same window index in order of cycle indexes; and
step F: constituting tensor data of MS2 fragment ion intensity from the data processed in step E based on three dimensions: a cycle index, a fragment ion mass-to-charge ratio, and a precursor ion window index corresponding to a fragment ion.

2. The implementation method of a molecular omics data structure based on data independent acquisition mass spectra according to claim 1, further comprising step G: pooling the data of different dimensions to reduce the size of the tensor data and then generating pooled DIAT data.

3. The implementation method of a molecular omics data structure based on data independent acquisition mass spectra according to claim 2, wherein the method of pooling in step G is: first, in each precursor isolation window, performing distribution statistical estimation on non-zero values of precursor ion mass-to-charge ratios to obtain a main and sub alternating peak mode with predefined grids; then pooling different mass-to-charge ratio areas by the pattern of the main and sub alternating peak mode, where the upper and lower boundaries of the mass-to-charge ratio areas were determined using nonlinear square Gaussian fitting of non-zero intensity distribution peaks; finally discarding all grids without peaks, and merging multiple rows of the main and sub peak areas into one row to reduce the rows in the mass-to-charge ratio dimension.

4. The implementation method of a molecular omics data structure based on data independent acquisition mass spectra according to claim 2, further comprising the following step: after obtaining the pooled DIAT data, processing the DIAT data into a pseudo-color image to achieve visualization.

5. The implementation method of a molecular omics data structure based on data independent acquisition mass spectra according to claim 2, further comprising the following step: after obtaining the pooled DIAT data, graying the fragment ion intensity in the DIAT data as an input model for deep learning.
